(19) United States
(12) Patent Application Publication
(10) Pub. No.: US 2025/0037595 A1
(43) Pub. Date: Jan. 30, 2025

(54) SYSTEM AND METHOD OF REMOTE REHABILITATION THERAPY FOR MOVEMENT DISORDERS

(71) Applicant: Encora, Inc., Boston, MA (US)

(72) Inventors: Daniel Carballo, Boston, MA (US); Allison Davanzo, Green Cove Springs, FL (US)

(21) Appl. No.: 18/786,359

(22) Filed: Jul. 26, 2024

Related U.S. Application Data
(60) Provisional application No. 63/516,280, filed on Jul. 28, 2023.

(51) Int. Cl. G09B 5/02; A61B 5/00; A61B 5/11; G16H 20/30; G16H 40/67

(52) U.S. Cl. CPC G09B 5/02; A61B 5/0002; A61B 5/11; A61B 5/4848; A61B 5/681; G16H 20/30; G16H 40/67; A61B 2562/0219

(57) ABSTRACT

A system for remote rehabilitation of a movement disorder of a patient is presented. A system includes a wearable device. A wearable device includes a sensor configured to generate movement data of a patient. Movement data is indicative of one or more movement disorder symptoms of a patient. A wearable device includes a processor in communication with a sensor. A wearable device includes a stimulator in communication with a processor. A processor is configured to cause a stimulator to stimulate a body part of a patient based on movement data. A wearable device includes a wireless communication unit in communication with a processor. A system includes a virtual platform running on a server in communication with a wearable device via a wireless communication unit. A virtual platform is programmed to receive movement data from a wearable device.

### CROSS REFERENCE TO RELATED APPLICATION

This application claims priority to U.S. Provisional Application No. 63/516,280, filed Jul. 28 2023, the entirety of which is incorporated herein by reference.

### TECHNICAL FIELD

The present disclosure relates to remote rehabilitation therapy. In particular, the present disclosure relates to systems and methods of remote rehabilitation therapy for movement disorders.

### SUMMARY

In an aspect, a system for remote rehabilitation of a movement disorder of a patient is presented. A system includes a wearable device. A wearable device includes a sensor configured to generate movement data of a patient. Movement data is indicative of one or more movement disorder symptoms of a patient. A wearable device includes a processor in communication with a sensor. A wearable device includes a stimulator in communication with a processor. A processor is configured to cause a stimulator to stimulate a body part of a patient based on movement data. A wearable device includes a wireless communication unit in communication with a processor. A system includes a virtual platform running on a server in communication with a wearable device via a wireless communication unit. A virtual platform is programmed to receive movement data from a wearable device. A virtual platform is programmed to host a provider application operable to run on a provider device. A provider application is programmed to generate a therapy regime based on movement data. A virtual platform is programmed to host a patient application operable to run on a patient device. A patient application is programmed to compare movement data to a therapy regime and provide real-time feedback to a patient through a patient device.

In another aspect, a method of remote rehabilitation of a movement disorder of a patient is presented. A method includes generating movement data by a sensor of a wearable device placed on a patient. Movement data is indicative of one or more movement disorder symptoms of a patient. A method includes communicating movement data from a wireless communication unit of a wearable device to a virtual platform running on a server. A method includes generating a therapy regime based on movement data by a provider application hosted by a virtual platform. A method includes comparing movement data to a therapy regime by a patient application hosted by a virtual platform. A method includes providing real-time feedback to a patient through a patient device in communication with a virtual platform via a patient application based on the comparison

The above and other preferred features, including various novel details of implementation and combination of elements, will now be more particularly described with reference to the accompanying drawings and pointed out in the claims. It will be understood that the particular methods and apparatuses are shown by way of illustration only and not as limitations. As will be understood by those skilled in the art, the principles and features explained herein may be employed in various and numerous embodiments.

### BRIEF DESCRIPTION OF THE DRAWINGS

The disclosed embodiments have advantages and features which will be more readily apparent from the detailed description, the appended claims, and the accompanying figures (or drawings). A brief introduction of the figures is below.

FIG. 1 is a block diagram of a system for remote rehabilitation therapy for movement disorders;

FIG. 2 illustrates a system for remote rehabilitation of movement disorders;

FIG. 3 illustrates a flowchart of a method of remote rehabilitation for movement disorders;

FIG. 4 is an illustration of a wearable device;

FIG. 5 is an exploded view of the wearable device of FIG. 4;

FIG. 6 is a block diagram of a machine learning module; and

FIG. 7 is a block diagram of a computing device.

### DETAILED DESCRIPTION

The Figures (Figs.) and the following description relate to preferred embodiments by way of illustration only. It should be noted that from the following discussion, alternative embodiments of the structures and methods disclosed herein will be readily recognized as viable alternatives that may be employed without departing from the principles of what is claimed.

Reference will now be made in detail to several embodiments, examples of which are illustrated in the accompanying figures. It is noted that wherever practicable similar or like reference numbers may be used in the figures and may indicate similar or like functionality. The figures depict embodiments of the disclosed system (or method) for purposes of illustration only. One skilled in the art will readily recognize from the following description that alternative embodiments of the structures and methods illustrated herein may be employed without departing from the principles described herein.

Aspects of the present disclosure may be used to facilitate remote rehabilitation of one or more movement disorder symptoms of a patient. For instance, a patient may have Parkinson's, Essential Tremor, Dystonia, Paralysis, Restless Leg Syndrome, Bradykinesia, Post-Stroke Hemiparesis or Spasticity, Cerebral Palsy, Spinal Cord Injury, Drug-Inducted Tremor, Freezing of Fait, Balance Disorders, Neuropathy, or any other movement disorder. A virtual platform may be used to allow for remote therapy regimes to be given to a patient. A provider may be able to adjust one or more parameters of stimulation of a wearable device remotely in accordance with embodiments described herein.

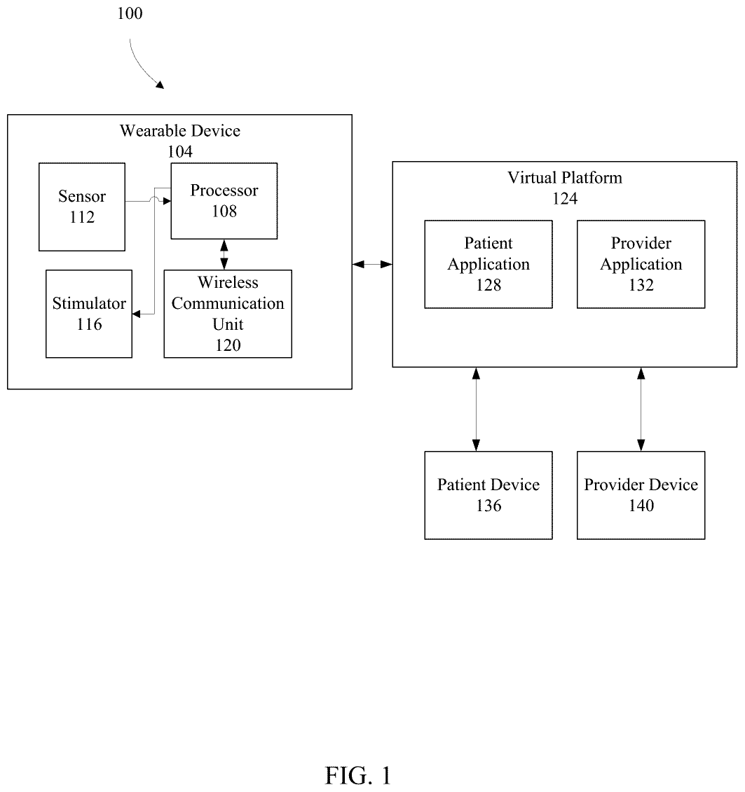

FIG. 1 illustrates a system 100 for remote rehabilitation of movement disorders. System 100 may include a wearable device 104. A “wearable device” as used in this disclosure refers to a device capable of stimulating a user's nerves that is attachable to the user. Wearable device 104 may take the form of, but is not limited to, wristbands, arm bands, leg bands, and/or other forms of wearable devices. In some embodiments, wearable device 104 may be a wrist-worn device. Wearable device 104 may include processor 108. Processor 108 may be in communication with a memory. A memory may include instructions configuring processor 108 to perform various tasks. Processor 108 may be in communication with sensor 112. A “sensor” as used throughout this disclosure is an element capable of detecting a physical property. Physical properties may include, but are not limited to, kinetics, electricity, magnetism, radiation, thermal energy, and the like. In some embodiments, wearable device 104 may include a sensor suite. A “sensor suite” as used throughout this disclosure is a combination of two or more sensors. Sensor 112 may have a plurality of sensors, such as, but not limited to, two or more sensors. Sensor 112 may have two or more of a same sensor type. In other embodiments, sensor 112 may have two or more differing sensor types. For instance, sensor 112 may include an electromyography sensor (EMG) and an inertial measurement unit (IMU). An IMU may be configured to detect and/or measure a body's specific force, angular rate, and/or orientation. Other sensors a sensor suite may include are accelerometers, gyroscopes, impedance sensors, temperature sensors, and/or other sensor types, without limitation. Sensor 112 may be configured to generate movement data. “Movement data” as used in this disclosure refers to any information pertaining to one or more movement disorder symptoms of a patient. Movement data may include, but is not limited to, accelerometer values, angular rotation values, EMG values, IMU values, tremor frequencies, tremor amplitudes, baseline tremor scores, and/or other values. Sensor 112 may be in communication with processor 108. A communication between the sensor 112 and processor 108 may be an electrical connection in which data may be shared between the sensor 112 and processor 108. In some embodiments, sensor 112 may be wirelessly connected to processor 108, such as through, but not limited to, a Wi-Fi, Bluetooth®, or other connection.

One or more sensors of sensor 112 may be configured to receive data from a patient, such as from a patient's body. Data received by one or more sensors of sensor 112 may include, but is not limited to, motion data, electric data, and the like. Motion data may include, but is not limited to, acceleration, velocity, angular velocity, and/or other types of kinetics. In some embodiments an IMU of sensor 112 may be configured to receive motion from a user's body. Motion may include, without limitation, vibration, acceleration, muscle contraction, and/or other aspects of motion. Motion may be generated from one or more muscles of a user's body. Muscle may include, but are not limited to, wrist muscles, hand muscles, forearm muscles, and/or other muscles. In an embodiment, motion generated from one or more muscles of a user's body may be involuntarily generated by one or more symptoms of a movement disorder of the user's body. A movement disorder may include, without limitation, Parkinson's disease (PD), post stroke recovery, and the like. Symptoms of a movement disorder may include, but are not limited to, stiffness, freezing of gait, tremors, shaking, involuntary muscle contraction, and/or other symptoms. In other embodiments, motion generated from muscles of a user's body may be voluntary. For instance, a user may actively control one or more of their muscles, which may generate motion that may be detected and/or received by sensor 112.

Still referring to FIG. 1, one or more sensors of sensor 112 may be configured to receive electrical data, such as the electrical activity that may be generated by one or more of muscles of a user. Electric data may include, but is not limited to, voltages, impedances, currents, resistances, reactance values, waveforms, and the like. For instance, electrical activity may include an increase in current and/or voltage of one or more of muscles during a contraction of the one or more of the muscles. An EMG of the sensor suite 112 may be configured to receive and/or detect electrical activity generated by one or more muscles of a user. In some embodiments, one or more sensors of sensor 112 may be configured to generate sensor output. “Sensor output” as used in this disclosure is information generated by one or more sensing devices. Sensor output may include, but is not limited to, voltages, currents, accelerations, velocities, and/or other output. Sensor output generated from one or more sensors of sensor 112 may be communicated to the processing unit 104, such as through a wired, wireless, or other connection. Processor 108 may be configured to determine a symptom of a movement disorder based on sensor output received from one or more sensors. Processor 108 may be configured to determine symptoms such as, but not limited to, stiffness, tremors, freezing of gait, and the like. Freezing of gait refers to a symptom of Parkinson's disease in which a person with Parkinson's experiences sudden, temporary episodes of inability to step forward despite an intention to walk. An abnormal gait pattern can range from merely inconvenient to potentially dangerous, as it may increase the risk of falls. Stiffness may refer to a muscle of a person with Parkinson's disease that may contract and become rigid without the person wanting it to. The processing unit 104 may compare one or more values of sensor output from sensor 112 to one or more values associated with one or more symptoms of a movement disorder. For instance, processor 108 may compare sensor output of one or more sensors of sensor 112 to one or more stored values that may already be associated with one or more symptoms of a movement disorder. As a non-limiting example, acceleration of a user's arm of about 1 in/s to about 3 in/s may correspond to a symptom of a light tremor. In some embodiments, wearable device 104 may be the same as the wearable device described in U.S. application Ser. No. 16/563,087, filed Sep. 6, 2019, and titled “Apparatus and Method for Reduction of Neurological Movement Disorder Symptoms Using Wearable Device”, the entirety of which is incorporated herein by reference.

Wearable device 104 may include stimulator 116. A “stimulator” as used throughout this disclosure refers to a device capable of emitting energy to stimulate a user's nerves and/or tissues. Stimulator 116 may include, but is not limited to, a vibratory, electric, thermal, or ultrasonic stimulation device. Wearable device 104 may include two or more stimulators 116. In some embodiments, wearable device 104 may include two or more stimulators 116 of differing types, such as a mechanical stimulator and an electrical stimulator, an electrical stimulator and an ultrasonic stimulator, and the like. Stimulators 116 may be positioned to provide stimulus to specific parts of a user's body. For instance, wearable device 104 may include one or more stimulators 116 that may be positioned to stimulate one or more portions of a user's peripheral nervous system. A “peripheral nervous system” as used in this disclosure refers to the part of nervous system that is outside the central nervous system (CNS). The peripheral nervous system may include one or more nerves and/or tissues. The peripheral nervous system may include one or more ganglion. “Ganglion” refers to a group of neuron cells bodies in the peripheral nervous system. Ganglion may include dorsal root ganglia and/or trigeminal ganglia. Nerves and/or tissues of a peripheral nervous system may be referred to as “peripheral nerves” and “peripheral tissues,” respectively. Processor 108 may be configured to target peripheral nerves and/or tissues of a peripheral nervous system of a user. Peripheral nerves and/or tissues may be located in a user's wrist, arm, neck, and/or other parts of a user. In some embodiments, stimulation may be delivered to one or more peripheral nerves and/or tissues of a user. In some embodiments, stimulation may be provided to one or more mechanoreceptors of a user's body. Mechanoreceptors” as used throughout this disclosure refer to cells of a human body that respond to mechanical stimuli. Mechanoreceptors may include proprioceptors and/or somatosensors. Proprioceptors may include head stems of muscles innervated by the trigeminal nerve. Proprioceptors may be part of one or more areas of a user's limbs, such as, but not limited to, wrists, hands, legs, feet, arms, and the like. Somatosensors may include cells having receptor neurons located in the dorsal root ganglion.

In some embodiments, one or more stimulators 116 may be positioned along a wristband of wearable device 104. Wearable device 104 may include, in an embodiment, four or more stimulators 116 that may be positioned within a wristband of wearable device 104. In other embodiments, one or more stimulators 116 may be positioned on a surface of a housing of wearable device 104. Stimulator 116 may include, but is not limited to, a piezoelectric motor, electromagnet motor, linear resonant actuator (LRA), eccentric rotating mass motor (ERMs), and the like. Stimulator 116 may be configured to vibrate at up to or more than 200 kHz, in an embodiment. Stimulator 116 may draw energy from one or more batteries from wearable device 104. For instance, one or more stimulators 116 may draw about 5 W of power from a battery of wearable device 104. In some embodiments, one or more stimulators 116 may have a max current draw of about 90 mA, a current draw of about 68 mA, a 34 mA current draw at 50% duty cycle, and may have a voltage of about 0V to about 5V, without limitation. In some embodiments, a suprasensory vibration produced by one or more stimulators 116 may have an acceleration greater than or equal to 50 mGᵣₘₛ. In some embodiments, a suprasensory vibration produced by one or more stimulators 116 may have an acceleration between 180 mGᵣₘₛ and 1.8Gᵣₘₛ. In some embodiments, a subsensory vibration produced by one or more stimulators 116 may have an acceleration between 0 and 50 mGᵣₘₛ.

Still referring to FIG. 1, processor 108 may be configured to command stimulator 116 to apply a stimulation to one or more mechanoreceptors of the users body. Stimulation produced by one or more stimulators 116 may include a waveform output calculated by processor 108. Stimulation may be applied to mechanoreceptors or other peripheral nerves and/or tissues, which may cause the mechanoreceptors to generate one or more afferent signals. An “afferent signal” as used in this disclosure is a neuronal signal in a form of action potentials that are carried toward target neurons. Afferent signals may be communicated to the peripheral nervous system (PNS) of a user's body. A user's brain may communicate efferent signals to the PNS 172 through the spinal cord. “Efferent signals” as used in this disclosure are signals that carry motor information for a muscle to take an action. Efferent signals may include one or more electrical signals that may cause one or more muscles to contract or otherwise move. For instance, the PNS may input afferent signals and communicate the afferent signals to the brain through the spinal cord. The brain may generate one or more efferent signals and communicate the efferent signals to the PNS through the spinal cord. The PNS may communicate the efferent signals to the muscles.

In some embodiments, stimulator 116 may be configured to stimulate various types of sensory fibers and afferent nerves, such as, but not limited to, Aδ fibers, C fibers, Ia afferents, Ib afferents, Aβ fibers, Aγ fibers, or dorsal root ganglion (DRG) neurons. In some embodiments, wearable device 104 may be configured to specifically target Aδ fibers and C fibers, which are known to transmit acute and chronic sensations of pain, respectively. Processor 108 may utilize a frequency of stimulation that may be specifically tailored to these fiber types. For instance, Aδ fibers may respond to a frequency range of about 2 Hz to about 200 Hz, while C fibers may respond to a range of about 0.5 to about 2 Hz. In some embodiments, wearable device 104 may target Ia and Ib afferents, by employing a frequency of stimulation specifically tailored to these afferents, such as at about 80 Hz to about 120 Hz. In some embodiments, wearable device 104 may be configured to target Aβ fibers, which are sensory nerve fibers that transmit touch and pressure sensations. For instance, a frequency of stimulation specifically tailored to these fibers may be about 60 Hz to about 80 Hz. Wearable device 104 may be configured to specifically target Aγ fibers, which are part of the motor system and innervate intrafusal muscle fibers, playing a crucial role in muscle tone and reflexes. For instance, a stimulation output may employ a frequency of stimulation specifically tailored to these fibers, such as about 60 Hz to about 80 Hz for electrical stimulation, about 80 Hz to about 120 Hz for vibratory stimulation, and about 1 MHz to about 3 MHz for ultrasound stimulation. Wearable device 104 may be configured to target Dorsal Root Ganglion (DRG) neurons, which are sensory neurons that transmit sensory information from the periphery to the spinal cord. For instance, a stimulation output may employ a frequency of stimulation specifically tailored to these neurons, such as in a range of about 2 Hz to about 200 Hz. In some embodiments, high-definition transcutaneous electrical nerve stimulation (HD-tENS) may be used to focus stimulation on DRG neurons. In some embodiments, one or more stimulators 116 may take the form of one or more electrodes, which may be placed on skin of a patient over muscles where targeted nerves/tissues may be located. In some embodiments, to prevent targeting unwanted nerves and/or tissues, wearable device 104 may be configured to use a spatially focused stimulation technique. A spatially focused stimulation technique may include using multiple stimulators 116, which may all be a same type or may be differing, such as electrodes, actuators, ultrasonic, or other types of stimulators 116, arranged in specific patterns to focus stimulation on targeted nerves, fibers, and/or tissues.

In some embodiments, to avoid contraction of one or more muscles of a patient, wearable device 104 may provide a simulation output below a motor threshold, which may be a minimum intensity that causes a visible muscle contraction. In some embodiments, wearable device 104 may be configured to determine a motor threshold of a patient. For instance, one or more stimulators 116 may deliver a series of stimulation pulses with gradually increasing intensity, while processor 108 monitors patient's muscle activity using electromyography (EMG) of sensor 112. A motor threshold may be identified as an intensity at which a significant increase in EMG activity is observed. In some embodiments, once a motor threshold is determined, wearable device 104 may keep a stimulation output below a percentage value of the motor threshold, such as below about 90% or less of the motor threshold. In some embodiments, to avoid interference with normal functioning of muscles, wearable device 104 may be configured to use a modulation technique such as amplitude modulation for electrical and/or vibratory stimulation or intensity modulation for ultrasound stimulation, which may control a strength of each stimulation pulse of the stimulation. In some embodiments, wearable device 104 may include a temperature sensor that may measure a skin temperature of a patient. Processor 108 may be configured to stop stimulation upon a temperature reading of excessive heating via a temperature sensor of wearable device 104. Processor 108 may automatically monitor skin temperature of a patient and adjust one or more parameters of a stimulation output accordingly.

With continued reference to FIG. 1, processor 108 may act in a closed-loop system. For instance processor 108 may act in a feedback loop between the data generated from one or more muscles of a user and stimulation output generated by one or more stimulators 116. Further, a closed-loop system may extend through and/or to the PNS, central nervous system (CNS), brain, and the like of a user's body based on afferent signals and efferent signals. In some embodiments, processor 108 may be configured to act in one or more modes. For instance, processor 108 may act in a first and a second mode. A first mode may include monitoring movements of a user's body passively to detect one or more movement disorder symptoms above a threshold. A threshold may include a root mean squared acceleration of 100 mG or 500 mGA, in an embodiment. A threshold may be set by a user and/or determined through processor 108 based on historical data. Historical data may include sensor and/or stimulation output data of a user over a period of time, such as, but not limited to, minutes, hours, weeks, months, years, and the like. A threshold may include, without limitation, one or more acceleration, pressure, current, and/or voltage values. In some embodiments, upon a threshold being reached, processor 108 may be configured to act in a second mode in which processor 108 commands one or more stimulators 116 to provide a stimulation output to one or more peripheral nerves and/or tissues of a user.

In some embodiments, processor 108 may utilize a stimulation selection algorithm. A stimulation selection algorithm may input current stimulation parameters of stimulation output and/or extracted features of sensor data and through a model free policy optimization algorithm may generate new stimulation parameters. Model free policy optimization may include, but is not limited to, Argmin, Q-learning, neural networks, genetic algorithms, differential dynamic programming, iterative quadratic regulator, and/or guided policy search. Processor 108 may continuously update stimulation parameters of stimulation output utilizing a stimulation selection algorithm. For instance, new stimulation parameters may become current stimulation parameters in a subsequent cycle, and the processing unit 104 may repeat the stimulation selection algorithm.

In some embodiments, processor 108 may utilize a classifier or other machine learning model that may categorize sensor output to categories of symptoms of a movement disorder. A “classifier,” as used in this disclosure is a machine-learning model, such as a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. Processor 104 and/or another device may generate a classifier using a classification algorithm, defined as a processes whereby a processor derives a classifier from training data. Classification may be performed using, without limitation, linear classifiers such as without limitation logistic regression and/or naive Bayes classifiers, nearest neighbor classifiers such as k-nearest neighbors classifiers, support vector machines, least squares support vector machines, Fisher's linear discriminant, quadratic classifiers, decision trees, boosted trees, random forest classifiers, kernel estimation, learning vector quantization, and/or neural network-based classifiers.

With continued reference to FIG. 1, a classifier may be generated, as a non-limiting example, using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A) P(A)=P(B), where P(AB) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table.

Processor 108 may calculate a likelihood table by calculating probabilities of different data entries and classification labels. Processor 108 may utilize a naïve Bayes equation to calculate a posterior probability for each class. A class containing the highest posterior probability is the outcome of prediction. Naïve Bayes classification algorithm may include a gaussian model that follows a normal distribution. Naïve Bayes classification algorithm may include a multinomial model that is used for discrete counts. Naïve Bayes classification algorithm may include a Bernoulli model that may be utilized when vectors are binary.

With continued reference to FIG. 1, a classifier may be generated using a K-nearest neighbors (KNN) algorithm. A “K-nearest neighbors algorithm” as used in this disclosure, includes a classification method that utilizes feature similarity to analyze how closely out-of-sample-features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing the classifier to select the k most similar entries training data to a given sample, determining the most common classifier of the entries in the database, and classifying the known sample. this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements.

A classifier may be trained with training data correlating motion data and/or electric data to symptoms of a movement disorder. Training data may be received through user input, external computing devices, and/or previous iterations of training. As a non-limiting example, an IMU of sensor 112 may receive motion data generated by one or more muscles of a user and may generate sensor output including acceleration values which may be communicated to processor 108. Processor 108 may classify and/or categorize sensor output to a symptom of freezing of gait.

Still referring to FIG. 1, processor 108 may train a classifier with training data correlating motion and/or electrical data to symptoms of a movement disorder. In other embodiments, training of a classifier and/or other machine learning model may occur remote from processor 108 and processor 108 may be sent one or more trained models, weights, and the like of a classifier, machine learning model, and the like. Training data may be received by user input, through one or more external computing devices, and/or through previous iterations of processing. A classifier may be configured to input sensor output, such as output of sensor 112, and categorize the output to one or more groups, such as, but not limited to, tremors, stiffness, freezing of gait, and the like.

Processor 108 may be configured to target various nerves and/or fibers through a machine learning model. For instance, a machine learning model may be trained with training data correlating symptoms and/or current stimulation parameters to one or more target nerves and/or fibers. Training data may be received via user input, external computing devices, and/or previous iterations of processing. A machine learning model may be trained to input data generated by sensor 112 and/or symptom data inferred by data generated by sensor 112 and output one or more parameters of a stimulation waveform or output that may target specific fibers and/or nerves. A machine learning model may be trained to adjust parameters of a stimulation waveform or output based on an identified target nerve and/or fiber. For instance and without limitation, a machine learning model may be trained to adjust stimulation parameters to target Aδ fibers, C fibers, Ia afferents, Ib afferents, Aβ fibers, Aγ fibers, and/or dorsal root ganglion (DRG) neurons. In some embodiments, processor 108 may utilize a classifier which may be trained to input data generated by sensor 112 and classify the data to one or more symptoms and/or targeted nerves and/or fibers. For instance, and without limitation, a classifier may be trained to classify one or more ranges of tremor waveforms to one or more specific target nerves and/or fibers. Training data may be received via user input, external computing devices, and/or previous iterations of processing. A classifier may be trained to categorize data generated by sensor 112 to classes of various fibers and/or nerves, such as, but not limited to, Aδ fibers, C fibers, la afferents, Ib afferents, Aβ fibers, Aγ fibers, and/or dorsal root ganglion (DRG) neurons. Based on data generated by sensor 112, processor 108, through utilization of one or more classifiers and/or machine learning models, may categorize the data to fibers and/or nerves predicted to be causing symptoms determined by the data. Determinations and/or predictions made by processor 108 and/or one or more classifiers and/or machine learning models may be used as training data to train one or more classifiers and/or machine learning models. Processor 108 may be configured to target one or more nerves and/of fibers predicted to have a highest impact on symptom severity. A highest impact may be in reference to one or more weights having a highest value that may be associated with each symptom severity of a plurality of symptoms. In some embodiments, patient application 128 and/or provider application 132 may be configured to utilize one or more machine learning models and/or classifiers as described above. In some embodiments, provider application 132 may be configured to identify target nerves and/or fibers, such as through use of one or more machine learning models and/or classifiers as described above, and provide a visualization of identified target nerves and/or fibers to a provider through provider device 140. A provider may adjust a therapy regime, stimulation parameters, and/or other data based on identified target fibers and/or nerves.

Processor 108 may calculate a stimulation output based on sensor output generated by one or more sensors of wearable device 104. A “stimulation output” as used in this disclosure is a signal having a frequency. A stimulation output may be generated as a vibrational, electrical, audial, and/or other output. A stimulation output may include one or more waveform outputs. A waveform output may include one or more parameters such as frequency, phase, amplitude, channel index, and the like. A channel index may include a channel of mechanoreceptors and/or of an actuator to be used. For instance, a channel index may include one or more channels of mechanoreceptors, actuators to stimulate the mechanoreceptors, and/or a combination thereof. The processing unit 104 may select one or more parameters of a waveform output based on received sensor output from one or more sensors of sensor 112. In other embodiments, waveform parameters of a stimulation output may be selected by the user. As a non-limiting example, a user may select stimulation and/or waveform parameters of a stimulation output from a predefined list of waveforms using buttons or other interactive elements on wearable device 104. A predefined list of stimulation outputs may include one or more waveforms having various frequencies, amplitudes, and the like, without limitation. A predefined list of stimulation outputs may be generated through previous iterations of stimulation output generation. In other embodiments, a predefined list of stimulation outputs may be entered by one or more users. In some embodiments, a predefined list of stimulation outputs may include stimulation outputs for specific symptoms, such as, but not limited to, freezing of gait, tremors, stiffness, and the like. In some embodiments, a user may select specific waveform parameters using an external computing device such as, but not limited to, a smartphone, laptop, tablet, desktop, smartwatch, and the like, which may be in communication with processor 108 through wireless communication unit 120.

In some embodiments, an output provided by one or more stimulators 116 may be subsensory. “Subsensory” refers to a level of stimulation not perceptible by a patient. In some embodiments, a stimulation output may suprasensory. “Suprasensory” refers to a level of stimulation perceptible by a user. In some embodiments, stimulator 116 may provide a suprasensory stimulation output that may indicate to a patient that stimulation is active. In some embodiments, wearable device 104 may include a display device and/or touchscreen and may allow a patient to adjust an intensity of a stimulation to ensure that the stimulation is comfortable for the patient. In some embodiments, wearable device 104 may include one or more buttons that may allow for an increase or decrease in stimulation intensity. Processor 108 may be configured to implement a feedback mechanism that allows a patient to indicate if stimulation is causing discomfort, and the intensity may be automatically adjusted based on this feedback.

A stimulation output provided by wearable device 104 may be patterned, in some embodiments. For instance a stimulation output may follow a specific sequence or rhythm, may be stochastic, random, or unpredictable. For instance, a stimulation output of wearable device 104 may have a continuous stimulation at a constant rate, such as at about 100 Hz. In some embodiments, a stimulation output may take form of a burst stimulation, which may have about 5 bursts of pulsed stimulation at a frequency of about 100 Hz with a gap of about 500 ms in between pulses. In some embodiments, a burst frequency may be about 40 Hz. In some embodiments, a stimulation output may be a high-frequency stimulation. A high frequency stimulation may include a stimulation output having a frequency of about 10 KHz or higher. In some embodiments, a stimulation output may be a low frequency stimulation. A low frequency stimulation may include a stimulation output having a frequency of about 2 Hz or less than about 2 Hz. In some embodiments, a stimulation output may be modulated. For instance, frequency, pulse width, amplitude, or other parameters of a stimulation output may vary over time. As a non-limiting example, a modulated stimulation output may have a frequency of about 40 Hz and a modulation depth of about 50%.

Still referring to FIG. 1, wearable device 104 may include wireless communication unit 120. A “wireless communication unit” as used throughout this disclosure is any form of software and/or hardware capable of transmission of electromagnetic energy. For instance, wireless communication unit 120 may be configured to transmit and receive radio signals, Wi-Fi signals, Bluetooth® signals, cellular signals, and the like. Wireless communication unit 120 may include a transmitter, receiver, and/or other component. A transmitter of wireless communication unit 120 may include, but is not limited to, an antennae. Antennas of wireless communication unit 120 may include, without limitation, dipole, monopole, array, loop, and/or other antenna types. A receiver of wireless communication unit 120 may include an antenna, such as described previously, without limitation. Wireless communication unit 120 may be in communication with processor 108. For instance, processor 108 may be physically connected to wireless communication unit 120 through one or more wires, circuits, and the like. Processor 108 may command wireless communication unit 120 to send and/or receive data transmissions to one or more other devices. For instance, and without limitation, wireless communication unit 120 data generated by sensor 112.

With continued reference to FIG. 1, system 100 may include virtual platform 124. A “virtual platform” as used in this disclosure refers to software, hardware, or a combination thereof, that facilitates communication between two or more devices. Virtual platform 124 may operate on a server, cloud-based network, or other computing device. Virtual platform 124 may be in communication with wearable device 104 via wireless communication unit 120. For instance in some embodiments, virtual platform 124 may be in communication with wireless communication unit 120 over a Wi-Fi or Cellular communication. In some embodiments, virtual platform 124 may be in communication with an external computing device, such as patient device 136 and/or provider device 140. Patient device 136 and/or provider device 140 may be in communication with wireless communication unit 120 and/or virtual platform 124 and may communicate data from wireless communication unit 120 to virtual platform 124, such as via Wi-Fi, Cellular, or other connections. For instance, Wi-Fi- or Li-Fi may be used for medium-range communication, Bluetooth may be used for short-range communication, and 4G, 5G, or 6G, may be used for long-range communication. Connections between virtual platform 124, wearable device 104, patient device 136, and/or provider device 140 may be encrypted. For instance and without limitation, one or more of Wi-Fi protected Access (WPA), Bluetooth encryption, KASUMI, SNOW, AES, OAUth, JWT, TLS, and/or other forms of encryption may be used. In some embodiments, accessing provider application 132 and/or patient application 128 may include two-factor authentication, GPS location confirmation, IP address confirmation, biometric authentication, and/or other forms of authentication.

Virtual platform 124 may be programmed to host one or more applications. For instance, virtual platform 124 may be programmed to host patient application 128 and/or provider application 132. A “patient application” as used in this disclosure is patient-facing software. A “provider application” as used in this disclosure is provider-facing software. Patient application 128 and/or provider application 132 may be coded in cross-platform frameworks, such as React Native or Flutter, in some embodiments. Provider application 132 may be operable to run on provider device 140. A “provider device” as used in this disclosure refers to a computing device used by a medical provider. A medical provider may be a nurse, doctor, or other licensed professional. Provider device 140 may include, but is not limited to, smartphones, tablets, desktops, laptops, and/or other devices. In some embodiments, provider application 132 may run locally on provider device 140. In other embodiments, provider application 132 may run on virtual platform 124 and may be accessible via provider device 140 which may be in communication with virtual platform 124. Provider application 132 may be configured to receive movement data and/or other data generated by wearable device 104 and communicated to virtual platform 124. Data generated by wearable device 104 may be communicated to virtual platform 124 directly via wireless communication unit 120 and/or indirectly via patient device 136 and/or provider device 140. Provider application 132 may be configured to receive data generated by wearable device 104 and generate a therapy regime based on the data. A “therapy regime” as used in this disclosure is a collection of one or more treatments to aid in reduction of symptom severity of one or more movement disorders. Symptoms of one or more movement disorder may include, but are not limited to, tremors, rigidness, freezing gait, and/or other movement disorder symptoms. A therapy regime may include stimulation provided by wearable device 104. In some embodiments, a therapy regime may include one or more exercise aimed at targeting one or more tissues/nerves that may be experiencing symptoms of a movement disorder. For instance and without limitation, exercise may include, but are not limited to, lateral raises, wrist and/or forearm flexion, shoulder presses, grip strength exercise, and/or other exercise. In some embodiments, a therapy regime may include stimulating one or more parts of a user's body while the user performs one or more exercises.

Provider application 132 may be configured to generate and/or suggest a therapy regime. For instance, provider application 132 may include a machine learning model that may be trained with training data correlating movement data and/or symptoms to therapy regimes. Training data may be received via user input, external computing devices, and/or previous iterations of processing. In some embodiments, provider application 132 may be in communication with a machine learning model that may be run external to provider application 132. In other embodiments, provider application 132 may include a machine learning model. A machine learning model of provider application 132 may be configured to input movement data and/or symptom data and output one or more parameters of a therapy regime. For instance and without limitation, parameters of a therapy regime may include targeted nerves and/or tissues, frequencies of waveform outputs, durations of waveform outputs, quantity of consecutive waveform outputs, exercise type, exercise repetitions, exercise duration per repetition, sets of repetitions, combinations of stimulation with exercises, and/or other parameters. Provider application 132 may be configured to store a plurality of patient data and corresponding therapy regimes across a variety of patient demographics and/or movement disorder symptoms. Provider application 132 correlate a current patient's demographics and/or movement disorder symptoms with one or more of a plurality of patient data and corresponding therapy regimes to determine an optimized therapy regime for the current patient. In some embodiments, optimization of a therapy regime may be performed using a machine learning model. In other embodiments, an objective function, loss function, or other function may be performed to calculate an optimal therapy regime. In some embodiments, provider application 132 may utilize a machine learning model to predict a progress of a patient's rehabilitation via a therapy regime. A machine learning model may be trained with training data correlating symptoms and/or treatment parameters to a patients progress of a therapy regime. Training data may be received via user input, external computing devices and/or previous iterations of processing.

Provider application 132 may present one or more therapy regimes to a provider via provider device 140. A provider may select one or more therapy regimes presented by provider application 132 via provider device 140. Upon selection of one or more therapy regimes via provider device 140, provider application 132 may communicate one or more therapy regimens with patient application 128. In some embodiments, a provider may start and/or stop activation of stimulator 116 and/or other parts of wearable device 104 remotely via provider device 140. For instance and without limitation, a provider may adjust one or more parameters of stimulation provided by stimulator 116 via provider application 132, which in turn may communicate one or more parameters to wearable device 104 via wireless communication unit 120. A provider may be able to override a current or future stimulation provided by wearable device 104 via provider application 132 based on input received by provider device 140. Input received by provider device 140, such as described above, may include, but is not limited to, touch screen input on a graphical user interface (GUI), mouse and keyboard input, virtual reality (VR) headset input, and/or any other forms of input a computing device may receive.

Still referring to FIG. 1, provider application 132 may be programmed to generate a visualization of movement or other data generated by wearable device 104. A “visualization” as used in this disclosure refers to a graphic, pictorial, or other image that represents data. For instance and without limitation, a visualization of movement data may include bar graphs, line graphs, waveform graphs, trending graphs of historical data, and/or other forms of visualization. In some embodiments, a provider may select various forms of visualization that may be provided by provider application 132. Provider application 132 may organize and store a plurality of patient data, therapy regimes, treatment devices, and/or other information and may allow a provider to access any of the prior listed information for each patient. In some embodiments, visualization may be facilitated by code libraries such as D3.js or Chart.js, without limitation.

Patient application 128 may be configured to run on patient device 136. A “patient device” as used in this disclosure is any computing device used by a patient. Patient device 136 may include, but is not limited to, smartphones, laptops, desktops, tablets, and/or other devices. In some embodiments, patient application 128 may be operable to run locally on patient device 136. In other embodiments, patient application 128 may run on virtual platform 124 and may be accessible to a patient via patient device 136. Patient application 128 may be configured to receive movement and/or other data generated by sensor 112 and/or processor 108 communicated to virtual platform 124. In some embodiments, patient application 128 may be configured to compare movement data and/or other data with one or more parameters of a therapy regime, such as a therapy regime generated by provider application 132, and provide a patient with real-time feedback. “Real-time” as used in this disclosure refers to an instantaneous or near-instantaneous time period with respect to a reference point. For instance, real-time may be with a range of about 0.1 to about 0.5 seconds, less than about 0.1 seconds, or other time periods. Feedback generated by patient application 128 may include a visualization of one or more exercises that may be part of a therapy regime. For instance and without limitation, a model of an arm, leg, shoulder, and/or whole body of an individual may be animated to visualize one or more exercises a patient may perform in a therapy regime.

In some embodiments, patient application 128 may generate a completion score of one or more exercises of a therapy regime based on movement and/or other data. For instance, sensor 112 of wearable device 104 may be configured to provide a real-time spatial estimate of a location of a user's body part, such as a leg, arm, wrist, shoulder, and/or other body part. Patient application 128 may calculate a real-time positioning of a user's body part based on data generate by sensor 112 and may compare the positioning to an ideal positioning under a therapy regime. Patient application 128 may be configured to guide a patient through one or more exercises of a therapy regime. For instance, patient application 128 may guide a patient through one or more exercises via displaying an animation of one or more exercises through a display of patient device 136. Patient application 128 may display a comparison of a real-time positioning of a patient's body part overlayed on a silhouette of an avatar performing an exercise, in some embodiments. In an embodiment where a silhouette is displayed, a coloring of the silhouette may represent an accuracy of a patient's movement with relation to an exercise of a therapy regime. For instance and without limitation, a green silhouette may represent an accurate movement of a patient's body part, a yellow silhouette may represent a movement of a patient's body part with some error, and a red silhouette may represent a movement of a patient's body part that does not match an exercise being displayed.

In some embodiments, patient application 128 may utilize a form of a computer vision model in combination with a camera of patient device 136. A computer vision model may be a machine learning model trained to identify and/or classify objects and/or other areas of interest in an image. In some embodiments, a computer vision model used by patient application 128 may be trained to identify a positioning of a patient's body part with respect to a three-dimensional space based on a two-dimensional image. In some embodiments, patient device 136 may include a depth sensor, which may allow patient application 128 to more accurately estimate a positioning of a patient's body part. Patient application 128 may utilize a depth sensor of patient device 136 and/or a computer vision model to identify a positioning of a patient's body part. Patient application 128 may compare a poisoning of a patient's body part with one or more exercise of a therapy regime. For instance, patient application 128 may be programmed to count a number of repetitions, sets, arm and/or leg angles, and/or other data of a patient performing an exercise.

Patient application 128 and/or provider application 132 may be programmed to generate an indication of an effectiveness of a therapy regime. An indication may include a visual, audial, and/or mechanical indication. An effectiveness of a therapy regime may be calculated by patient application 128 and/or provider application 132 based on current therapy regime parameters, stimulation outputs, and/or movement or other data generated by wearable device 104. Various levels of effectiveness may be calculated. For instance a low effectiveness may be calculated for reduction in symptom severity of about 20% to about 30%, a medium effectiveness may be calculated for reduction in symptom severity of about 30% to about 50%, and a high effectiveness may be calculated for reduction in symptom of about 50% or greater, without limitation. Indications may take form of icons displayed on graphical user interfaces of patient device 136 and/or provider device 140, speaker sounds generated by patient device 136 and/or provider device 140, and/or vibratory and/or audible indications generated by a motor and/or speaker of wearable device 104. In some embodiments, wearable device 104 may provide periodic suprasensory stimulation bursts, an indicator sound that signals the operation of the device, an indicator light that provides a visual cue, a combination of concurrent subthreshold and suprathreshold signals produced through different stimulators within wearable device 104, and/or or a superposition of subthreshold and suprathreshold signals.

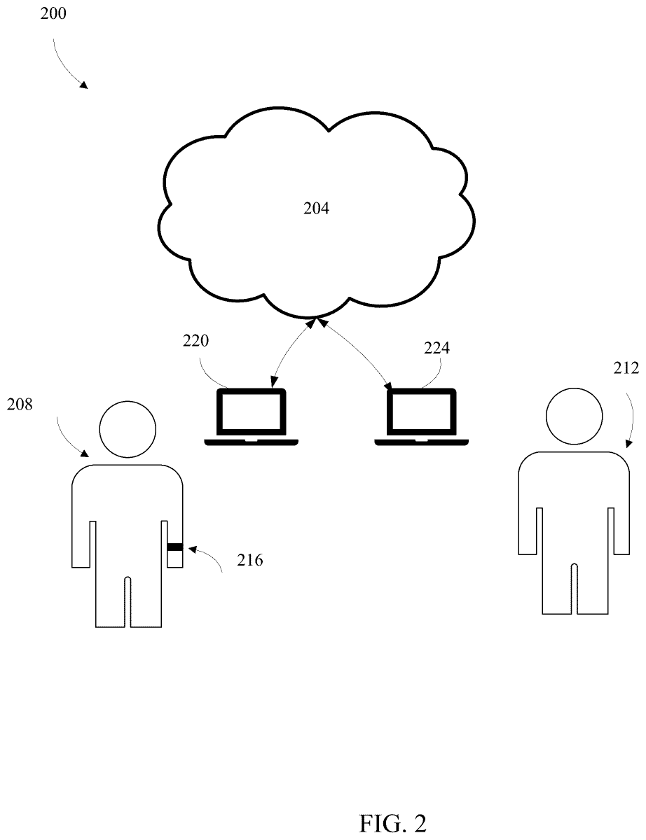

Referring now to FIG. 2, an illustration of a video call 200 performed by a virtual platform is presented. In some embodiments, wearable device 216 may act as a login token. For instance, wearable device 216 may provide virtual platform 204 with one or more authorization tokens that may be generated based on GPS locations, IP addresses, biometric information, and/or unique identification of wearable device 216. Unique identification of wearable device 216 may include, but is not limited to, identification numbers and/or strings unique to wearable device 216, unique circuit output of wearable device 216, radio and/or cellular signals uniquely encoded to wearable device 216, and/or other unique identifications of wearable device 216. In some embodiments, patient 208 may interact with one or more interactive elements of wearable device 216, which may include biometric identifiers, that may provide access of virtual platform 204 to patient 208. As a non-limiting example, patient 208 may provide user input to wearable device 216, which may directly communicate an authorization token to virtual platform 204 or may indirectly communicate an authorization token to patient device 220, which may forward the authorization token to virtual platform 204. Video call 200 may take place over virtual platform 204 between patient 208 and provider 212. Patient 208 may be wearing wearable device 216. Virtual platform 204 and wearable device 216 may be as described above with reference to FIG. 1, without limitation. In some embodiments, virtual platform 204 may be in communication with patient device 220 and provider device 224, each of which may be as described above with reference to FIG. 1. In some embodiments, wearable device 216 may be in communication with virtual platform 204 directly or via patient device 220. Each of patient device 220 and provider device 224 may include a camera and/or microphone, which may facilitate a video call between patient 208 and provider 212. Provider 212 may have access to data of patient 208 via a provider application, such as described above with reference to FIG. 1. Patient 208 may log into a patient application and provider 212 may log into a provider application, each of which may communicate with each other via virtual platform 204. Provider 212 may adjust one or more settings or parameters of wearable device 216 in real-time via provider device 224 and virtual platform 204. In some embodiments, provider 212 may provide a therapy regime to patient 208 in real-time via virtual platform 204. One or more parameters of a therapy regime may be presented to patient 208 via a patient application displayed through patient device 220. Patient 220 may perform one or more exercises in real-time which may be displayed to provider 212 through provider device 224. In some embodiments, a provider application may display both a real-time video call of patient 208 and data such as movement data, therapy regime data, or other data simultaneously.

In some embodiments, during, before, or after video call 200 patient 208 may be assessed for one or more movement disorder symptoms. Patient 208 may fill out a questionnaire via patient device 220 and/or a GUI of wearable device 216. Patient 212 may rate their symptoms on a scale, such as the Unified Parkinson' Disease Rating Scale (UPDRS). A patient application may prompt patient 220 to complete a UPDRS assessment at intervals throughout a day, week, month, or other time periods. A UPDRS assessment may be compared to data generated by one or more sensors as described below to prevent bias or inaccuracy. For instance and without limitation, accelerometry, electromyography, infrared thermography, 3-D motion capture systems, and/or other systems may be used to generated data to compare UPDRS scale ratings to. Data may be entered into a patient application and sent to a provider application via virtual platform 204.

In some embodiments, patient device 220 may include a 3-D motion capture system. A 3-D motion capture system may include a depth sensor, multiple cameras, structure light camera, laser triangulation system, time-of-flight camera, and/or other devices. A 3-D motion capture system may be configured to determine a positioning of a patient's body. For instance and without limitation, gait analysis of a patient may be performed. A patient's walking pattern may be analyzed using a 3-D motion capture system which may provide an objective measure of gait abnormalities. In some embodiments, wearable device 216 may include an infrared sensor, which may be configured to measure patient's 208 skin temperature. A measurement of patient's 208 skin temperature may provide an objective measure of autonomic symptoms, such as sweating or flushing. A reference temperature sensor may be used to calibrate an infrared sensor. In some embodiments, wearable device 216 may include an EMG system, which may provide an objective measure of muscle activity. Wearable device 216 may include an accelerometer, which may provide an objective measure of motor symptoms, such as, but not limited to, tremor or bradykinesia. As described above, a machine learning model and/or classifier may be used to classify various symptoms based on data generated by any device described throughout this disclosure without limitation.

Referring still to FIG. 2, in some embodiments, during, before, of after video call 200 one or more physical therapy exercises may be performed. Physical therapy exercises may be paired with a patient application that may provide instructions for range of motions exercises, such as, but not limited to, shoulder circles or ankle pumps. A patient application may provide visual and/or audio instructions for each exercise of one or more exercises. Physical therapy exercises may include strengthening exercises, such as leg lifts, arm curls, shoulder press, and/or other exercises, without limitation. In some embodiments, physical therapy exercises may include balance exercises, such as standing on one leg, or walking heel-to-toe, without limitation. In some embodiments, physical therapy exercises may include aerobic exercises such as walking or cycling, without limitation. In some embodiments physical therapy exercise may include flexibility exercises, such as stretching or yoga, without limitation.

In some embodiments, wearable device 216 may include one or more user interface components, such as, but not limited to, haptic feedback motors, light emitting diode (LED) indicators, speakers, displays, touchscreen interfaces, or other components. For instance, a haptic feedback motor may be used to provide vibration and/or movements to notify a patient user wearable device 216 is active or when a specific threshold is reached. Specific thresholds may include, but are not limited to, durations of stimulation, stimulation intensities, and/or other values. Haptic feedback may include a vibration of about 200 Hz for about 500 ms, in an embodiment. Haptic feedback may be adjusted via user input. In some embodiments, LED indicators may be used to emit light signals indicative of a status of wearable device 216. For instance, a green light may be indicative of an active status and a red light may be indicative of low battery. LED brightness and/or colors may be adjusted via user input. In some embodiments, audio signals may be emitted by wearable device 216. Audio signals may correspond to various indications of wearable device 216, for instance a beep may be indicative of an active status and a voice alert may be used for low battery. Audio signals may be adjusted via user input. In some embodiments, wearable device 216 may include a display, such as a liquid crystal display (LCD) screen. A display may provide a status of wearable device 216 and/or a progress of a therapy regime, stimulation, and/or other parameters. A display may show information such as current stimulation parameters, duration of the therapy session, and/or patient 220's symptom scores. In some embodiments, wearable device 216 may include a touchscreen interface. A touchscreen interface may be positioned over a GUI with one or more buttons for starting and stopping a therapy, one or more slider icons for adjusting stimulation parameters, and/or menus for selecting different therapy modes. A touchscreen may be capacitive and/or may support multi-touch input.

In some embodiments, patient device 220 may include a VR or augmented reality (AR) system. In some embodiments, communications between patient 220 and provider 212 may occur live in a AR/VR environment. For instance and without limitation, an AR/VR system may provide patient 220 and/or provider 212 with a virtual avatar. Patient 220 and/or provider 212 may communicate with each other through one or more virtual avatars. In some embodiments, an AR/VR system may present patient 220 with a pre-recorded instructional video corresponding to performing one or more exercises. A pre-recorded instructional video may include a 2D or 3D video, which may be interacted with by patient 220 through an AR/VR system. A patient application may be programmed to generate one or more virtual environments for patient 220 to perform one or more steps of a therapy regime in. For instance and without limitation, virtual environments of a patient application may include 3-D icons, graphics, avatars, alerts, and/or other computerized imagery. In some embodiments, an avatar of a patient performing one or more exercise may be presented in a VR environment to patient 204 via a VR headset of patient device 220. In some embodiments, AR may be used, which may overlay one or more computerized images over an immediate surrounding of patient 204. In some embodiments, through a display screen or through VR and/or AR, real-time feedback may be provided to patient 220. Real-time feedback may include directional guidance of one or more exercise of a therapy regime. Directional guidance may include two-dimensional and/or three-dimensional icons, graphics, avatars, and the like, that may indicate to patient 220 how to perform one or more exercises. For instance and without limitation, indications on how to perform one or more exercises of a therapy regime may include increasing a range of motion, performing multiple repetitions/sets, increase a duration of an exercise, adjusting a pathway of one or more body part's of patient 220 through an ideal pathway for performing one or more exercises, and/or other indications. In some embodiments, real-time feedback may include one or more audio signals. For instance and without limitation, patient device 220 and/or wearable device 216 may produce beeps, chirps, voice notes, and/or other audio signals that may be indicative of a completion of one or more parameters of a therapy regime. In some embodiments, a visualization of one or more exercises of a therapy regime may be presented to patient 204 via patient device 220. Visualizations may include, but are not limited to, icons, graphics, avatars, animations, and/or other visualizations. For instance and without limitation, a visualization may include an avatar performing one or more exercises.

In some embodiments, an AR/VR environment may provide for a gamification of parameters of a therapy regime. For instance, patient 220 may be provided with one or more objectives corresponding to one or more therapy regime parameters, such as exercises. An AR/VR environment may provide patient 220 with scores of completion of one or more therapy regime parameters and/or may provide patient 220 with celebratory icons upon completion of one or more therapy regime parameters. An AR/VR environment may enable tracking of and/or provide visual information of exercise data, such as, but not limited to, movement patterns, repetition counts, repetition speeds, power output of an exercise repetition, muscle fiber recruitment, range of motion, blood oxygenation, respiration, and/or other data. For instance, an AR/VR environment may generate exercise data utilizing one or more tracking devices of an AR/VR system. In other embodiments, exercise data may be communicated externally to an AR/VR system and may be displayed in a AR/VR environment through the AR/VR system. Displaying exercise data may occur in real time, without limitation.

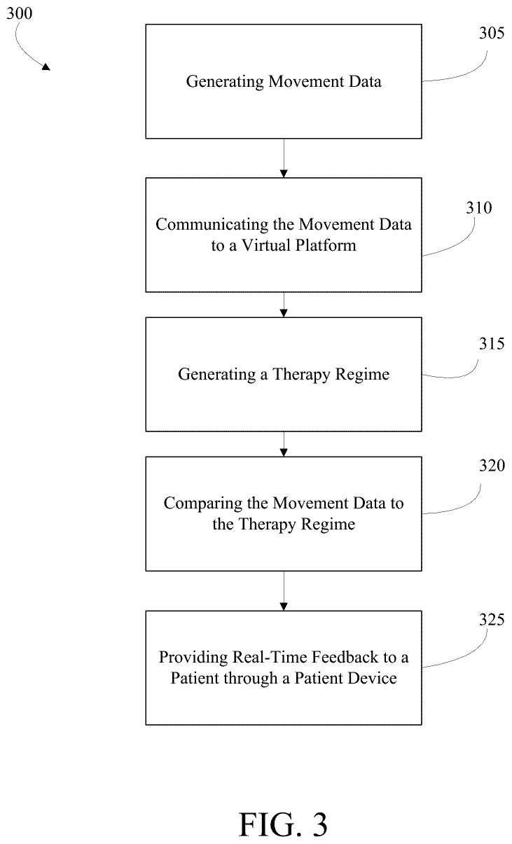

Referring now to FIG. 3, a method 300 of remote rehabilitation therapy for a movement disorder is presented. At step 305, method 300 includes generating movement data. Movement data may be generated by a sensor of a wearable device, such as described above with reference to FIG. 1. In some embodiments, movement data may be indicative of one or more movement disorder symptoms. Movement data may include, but is not limited to, accelerometer values, IMU values, gyroscope values, and/or other values. In some embodiments, movement data may include other physiological parameters of a patient, such as skin temperature, electrical activity of one or more muscles, and/or other parameters. This step may be implemented as described above with reference to FIGS. 1-2, without limitation.

At step 310, method 300 includes communicating the movement data to a virtual platform. The movement data may be communicated directly from a wearable device to a virtual platform. In some embodiments, movement data may be communicated to a patient device which may forward the movement data to a virtual platform. A virtual platform may be a cloud-based system, a server, and/or other computing infrastructure. A virtual platform may include a patient application and a provider application. In some embodiments, a virtual platform may be in communication with a patient device and a provider device. Communications between devices and a virtual platform may be encrypted, in some embodiments. This step may be implemented as described above with reference to FIGS. 1-2, without limitation.

At step 315, method 300 includes generating a therapy regime. A therapy regime may be generated by a provider application running on a virtual platform or locally on a provider device. In some embodiments, a therapy regime may include one or more parameters to assist a patient in reducing symptom severity of one or more movement disorders. Parameters of a therapy regime may include, but are not limited to, stimulation parameters, one or more rehabilitation exercises, and/or other parameters. In some embodiments, a therapy regime may be generated by a machine learning model. For instance and without limitation, a machine learning model may input movement data and/or patient demographic and/or symptom data and may output one or more parameters of a therapy regime. In other embodiments, a provider may select one or more parameters of a therapy regime via a provider application in communication with a virtual platform. In some embodiments, a provider application may generate one or more recommended therapy regimes for a patient, each with varying parameters, timelines for completion, estimates of symptom severity reduction, and/or other information. This step may be implemented as described above with reference to FIGS. 1-2, without limitation.

At step 320, method 300 includes comparing the movement data to a therapy regime. Comparisons may be made by a virtual platform and/or a provider application. Movement data may be compared to one or more threshold values that may represent symptom severity, such as tremor amplitude, tremor frequency, and/or other values. In some embodiments, a therapy regime may include performing one or more exercise while being stimulated by a wearable device or while being unstimulated. Movement data may be indicative of a positioning of a patient's body part and may be correlated to one or more exercises of a therapy regime. A comparison between a positioning of a patient's body part and an ideal positioning of a patient's body part with respect to one or more exercises may be made. This step may be implemented as described above with reference to FIGS. 1-2, without limitation.

At step 325, method 300 includes providing real-time feedback to a patient through a patient device. Real-time feedback may include visual, mechanical, and/or audial feedback. For instance and without limitation, real-time feedback may include displaying completion scores of one or more exercise through a patient device. In some embodiments, real-time feedback may include one or more audio signals, such as beeping from a wearable device, voice messages outputted from a wearable device of patient device, and/or other audio signals. In some embodiments, a provider may adjust one or more parameters of stimulation provided to a patient via a wearable device remotely. A provider may interact with a provider application hosted on a virtual platform via a provider device and may adjust stimulation parameters through the provider application which may be communicated to a wearable device via the virtual platform. This step may be implemented as described above with reference to FIGS. 1-2, without limitation.

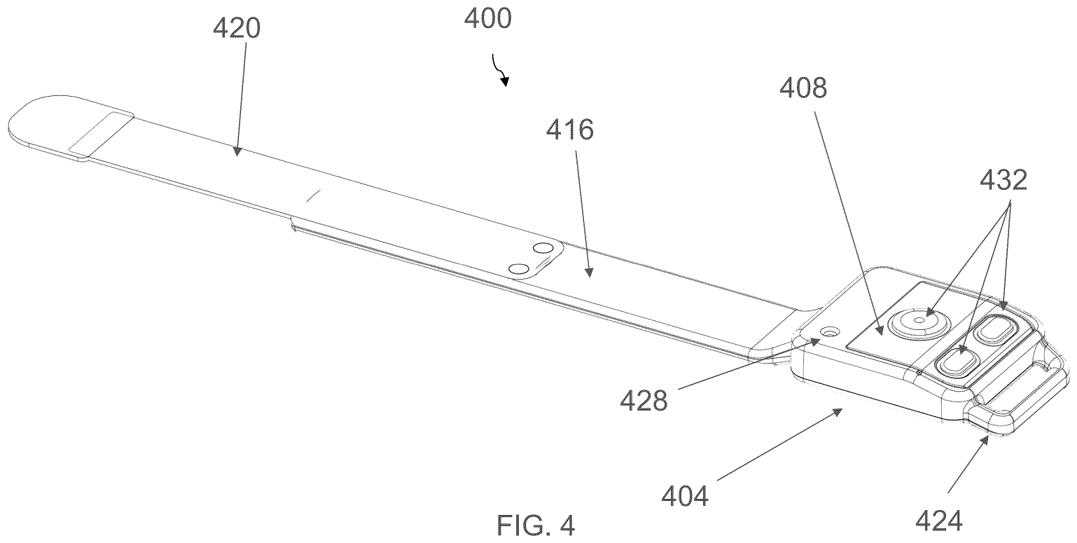

Referring now to FIG. 4, an illustration of a wearable device 400 is presented. Wearable device 400 may include housing 404. Housing 404 may be rectangular, circular, or other shapes. In some embodiments, housing 404 may be designed to house control module 408. Control module 408 may include a controller, processor, or other device. Control module 408 may include one or more resistors, transistors, capacitors, sensors, or other electrical components. Control module 408 may have one or more interactive elements 432. Interactive elements 432 may include, but are not limited to, buttons, touch sensors, capacitive sensors, and/or other devices. Interaction of one or more interactive elements 432 may cause a processor of control module 408 to perform various functions. In some embodiments, interactive elements 432 may include a power button and two or more stimulation adjusting buttons. A power button may turn on an off wearable device 400, enable a pairing mode of wearable device 400, or perform other functions. Stimulator adjusting buttons of interactive elements 432 may adjust one or more parameters of a stimulation output generated by control module 408. In some embodiments, interactive elements 432 may include a first stimulation adjuster button and a second stimulation adjuster button. A first stimulation adjuster button may be configured to increase one or more parameters of a stimulation output while a second adjuster button may be configured to decrease one or more parameters of a stimulation output. Stimulator adjuster buttons of interactive elements 432 may adjust any parameter of a stimulation output described throughout this disclosure, without limitation.

Control module 408 may be removably couplable to housing 404 and/or wristband 416. For instance, housing 404 may be designed as a snap-in case, which may allow an insertion of control module 408 into housing 404 via a snaping mechanism. Wristband 416 may extend away from housing 404 and may be designed to wrap around a portion of a user's body, such as a wrist or arm, without limitation. Wristband 416 may secure around a portion of a user's body via securing element 420. Securing element 420 may be a hook and loop fastener, a magnetic strap, Velcro, and/or other securing devices. Wristband 416 may secure itself to slot 424 of housing 404. Slot 424 may be shaped to allow a width of wristband 416 to pass through itself. In some embodiments, a user may adjust a tension of wristband 416 by adjusting an amount of length of wristband 416 passing through slot 424. As a non-limiting example, a user may wrap wristband 416 around their wrist and insert an end of wristband 416 into slot 424. A user may tension wristband 416 through slot 404 and secure wristband 416 to itself via securing element 420. Housing 404 may include light emitting diode (LED) 428. LED 428 may be placed on a top facing surface of housing 404. In some embodiments, LED 428 may be placed at a top left or top right side facing surface of housing 404. LED 428 may emit one or more wavelengths of light, which may indicate various information to a user. LED 428 may be configured to emit pulses of light.

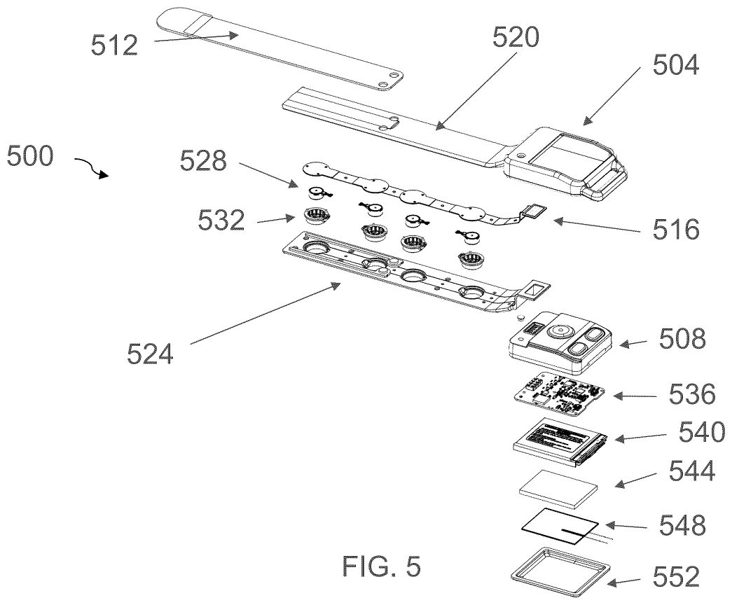

Referring now to FIG. 5, an exploded view of the wearable device of FIG. 4 is presented. Wearable device 500 may include housing 504, securing element 512, and control module 508, each of which may be as described above with reference to FIG. 4. Wearable device Wristband 500 may include a flexible printed circuit board (PCB) 516. Flexible PCB 516 may be positioned between top wristband half 520 and bottom wristband half 524. Flexible PCB 516 may be in electrical and/or mechanical communication with stimulators 528. Stimulators 528 may be, but are not limited to, electric, ultrasonic, heat, or vibratory. Wearable device 500 may include one or more motor caps 532, in embodiments where stimulators 528 may be vibratory. Control module 508 may include a main PCB 536, battery 540, foam spacer 544, charging coil 548, and/or a bottom housing component 552. Main PCB 536 may include processors, controllers, sensors, and/or other components. Main PCB 536 may be configured to connect to flexible PCB 516 via an electrical connection. Main PCB 536 may control one or more stimulators 528 via an electrical connection to flexible PCB 516. Battery 540 may be any type of battery, such as, but not limited to, lithium-ion, alkaline, or other batteries. Battery 540 may be configured to power main PCB 536, flexible PCB 516, stimulators 528, and/or other components. Battery 540 may be rechargeable via charging coil 548. Charging coil 548 may be configured to receive electrical power via electromagnetic induction. Charging coil 548 may provide power received via electromagnetic induction to battery 540. Foam spacer 544 may be positioned between battery 540 and charging coil 548, which may provide insulation to battery 540 from charging coil 548. Bottom housing component 552 may connect to a top housing component of control module 508. For instance, bottom housing component 552 may be placed underneath one or more components of control module 508 and may secure one or more components within an interior formed by a connection of bottom housing component 552 to a top housing component of control module 508.

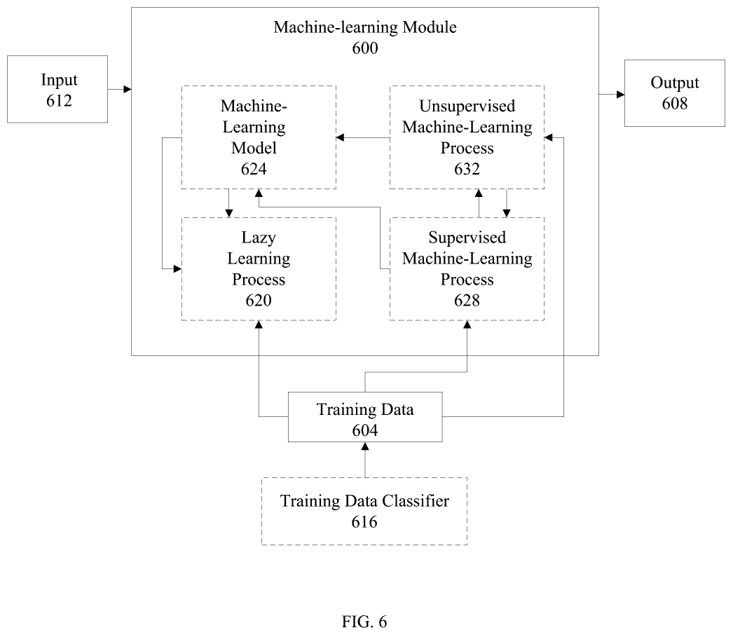

Referring to FIG. 6, an exemplary machine-learning module 600 may perform machine-learning process(es) and may be configured to perform various determinations, calculations, processes and the like as described herein using one or more machine-learning processes.

Machine learning module 600 may utilize training data 604. For instance, and without limitation, training data 604 may include a plurality of data entries, each entry representing a set of data elements that were recorded, received, and/or generated together. Training data 604 may include data elements that may be correlated by shared existence in a given data entry, by proximity in a given data entry, or the like. Multiple data entries in training data 604 may demonstrate one or more trends in correlations between categories of data elements. For instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 604 according to various correlations. Correlations may indicate causative and/or predictive links between categories of data elements, which may be modeled as relationships such as mathematical relationships by machine-learning processes as described in further detail below. Training data 604 may be formatted and/or organized by categories of data elements. Training data 604 may, for instance, be organized by associating data elements with one or more descriptors corresponding to categories of data elements. As a non-limiting example, training data 604 may include data entered in standardized forms by one or more individuals, such that entry of a given data element in a given field in a form may be mapped to one or more descriptors of categories. Elements in training data 604 may be linked to descriptors of categories by tags, tokens, or other data elements. Training data 604 may be provided in fixed-length formats, formats linking positions of data to categories such as comma-separated value (CSV) formats and/or self-describing formats. Self-describing formats may include, without limitation, extensible markup language (XML), JavaScript Object Notation (JSON), or the like, which may enable processes or devices to detect categories of data.

With continued reference to refer to FIG. 6, training data 604 may include one or more elements that are not categorized. Uncategorized data of training data 604 may include data that may not be formatted or containing descriptors for some elements of data. In some embodiments, machine-learning algorithms and/or other processes may sort training data 604 according to one or more categorizations. Machine-learning algorithms may sort training data 604 using, for instance, natural language processing algorithms, tokenization, detection of correlated values in raw data and the like. In some embodiments, categories of training data 604 may be generated using correlation and/or other processing algorithms. As a non-limiting example, in a body of text, phrases making up a number “n” of compound words, such as nouns modified by other nouns, may be identified according to a statistically significant prevalence of n-grams containing such words in a particular order. For instance, an n-gram may be categorized as an element of language such as a “word” to be tracked similarly to single words, which may generate a new category as a result of statistical analysis. In a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries in an automated fashion may enable the same training data 604 to be made applicable for two or more distinct machine-learning algorithms as described in further detail below. Training data 604 used by machine-learning module 600 may correlate any input data as described in this disclosure to any output data as described in this disclosure, without limitation.

Further referring to FIG. 6, training data 604 may be filtered, sorted, and/or selected using one or more supervised and/or unsupervised machine-learning processes and/or models as described in further detail below. In some embodiments, training data 604 may be classified using training data classifier 616. Training data classifier 616 may include a classifier. A “classifier” as used in this disclosure is a machine-learning model that sorts inputs into one or more categories. Training data classifier 616 may utilize a mathematical model, an artificial neural network, or a program generated by a machine learning algorithm. A machine learning algorithm of training data classifier 616 may include a classification algorithm. A “classification algorithm” as used herein is one or more computer processes that generate a classifier from training data. A classification algorithm may sort inputs into categories and/or bins of data. A classification algorithm may output categories of data and/or labels associated with the data. A classifier may be configured to output a datum that labels or otherwise identifies a set of data that may be clustered together. Machine-learning module 600 may generate a classifier, such as training data classifier 616 using a classification algorithm. Classification may be performed using, without limitation, linear classifiers such as without limitation logistic regression and/or naive Bayes classifiers, nearest neighbor classifiers such ask-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 616 may classify elements of training data to one or more parameters of a therapy regime.

Still referring to FIG. 6, machine-learning module 600 may be configured to perform a lazy-learning process 620 which may include a “lazy loading” or “call-when-needed” process and/or protocol. A “lazy-learning process” may include a process in which machine learning is performed upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 604. Heuristic may include selecting some number of highest-ranking associations and/or training data 604 elements. Lazy learning may implement any suitable lazy learning algorithm, including without limitation a K-nearest neighbors algorithm, a lazy naive Bayes algorithm, or the like. Persons skilled in the art, upon reviewing the entirety of this disclosure, will be aware of various lazy-learning algorithms that may be applied to generate outputs as described herein, including lazy learning applications of machine-learning algorithms as described in further detail below.

Still referring to FIG. 6, machine-learning processes as described herein may be used to generate machine-learning models 624. A “machine-learning model” as used herein is a mathematical and/or algorithmic representation of a relationship between inputs and outputs, as generated using any machine-learning process including without limitation any process as described above, and stored in memory. For instance, an input may be sent to machine-learning model 624, which once created, may generate an output as a function of a relationship that was derived. For instance, and without limitation, a linear regression model, generated using a linear regression algorithm, may compute a linear combination of input data using coefficients derived during machine-learning processes to calculate an output. As a further non-limiting example, machine-learning model 624 may be generated by creating an artificial neural network, such as a convolutional neural network comprising an input layer of nodes, one or more intermediate layers, and an output layer of nodes. Connections between nodes may be created via the process of “training” the network, in which elements from a training data 604 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 6, machine-learning algorithms may include supervised machine-learning process 628. A “supervised machine learning process” as used herein is one or more algorithms that receive labelled input data and generate outputs according to the labelled input data. For instance, supervised machine learning process 628 may include sensor data as described above as inputs, therapy regimes as outputs, and a scoring function representing a desired form of relationship to be detected between inputs and outputs. A scoring function may maximize a probability that a given input and/or combination of elements inputs is associated with a given output to minimize a probability that a given input is not associated with a given output. A scoring function may be expressed as a risk function representing an “expected loss” of an algorithm relating inputs to outputs, where loss is computed as an error function representing a degree to which a prediction generated by the relation is incorrect when compared to a given input-output pair provided in training data 604. Persons skilled in the art, upon reviewing the entirety of this disclosure, will be aware of various possible variations of at least a supervised machine-learning process 628 that may be used to determine relation between inputs and outputs. Supervised machine-learning processes may include classification algorithms as defined above.

Further referring to FIG. 6, machine learning processes may include unsupervised machine-learning processes 632. An “unsupervised machine-learning process” as used herein is a process that calculates relationships in one or more datasets without labelled training data. Unsupervised machine-learning process 632 may be free to discover any structure, relationship, and/or correlation provided in training data 604. Unsupervised machine-learning process 632 may not require a response variable. Unsupervised machine-learning process 632 may calculate patterns, inferences, correlations, and the like between two or more variables of training data 604. In some embodiments, unsupervised machine-learning process 632 may determine a degree of correlation between two or more elements of training data 604.

Still referring to FIG. 6, machine-learning module 600 may be designed and configured to create a machine-learning model 624 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of I divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model. Linear regression models may be generalized in an embodiment to polynomial regression models, whereby a polynomial equation (e.g. a quadratic, cubic or higher-order equation) providing a best predicted output/actual output fit is sought. Similar methods to those described above may be applied to minimize error functions, according to some embodiments.

Continuing to refer to FIG. 6, machine-learning algorithms may include, without limitation, linear discriminant analysis. Machine-learning algorithm may include quadratic discriminate analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include various forms of latent space regularization such as variational regularization. Machine-learning algorithms may include Gaussian processes, such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized tress, AdaBoost, gradient trec boosting, and/or voting classifier methods. Machine-learning algorithms may include neural net algorithms, including convolutional neural net processes

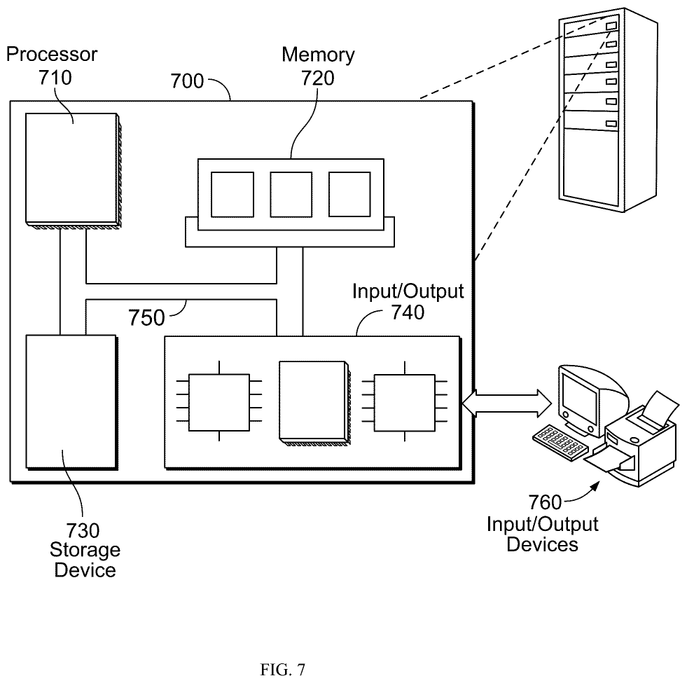

FIG. 7 is a block diagram of an example computer system 700 that may be used in implementing the technology described in this document. General-purpose computers, network appliances, mobile devices, or other electronic systems may also include at least portions of the system 700. The system 700 includes a processor 710, a memory 720, a storage device 730, and an input/output device 740. The apparatus may include disk storage and/or internal memory, each of which may be communicatively connected to each other. The apparatus 100 may include a processor 710. The processor 710 may enable both generic operating system (OS) functionality and/or application operations. In some embodiments, the processor 710 and the memory 720 may be communicatively connected. As used in this disclosure, “communicatively connected” means connected by way of a connection, attachment, or linkage between two or more elements which allows for reception and/or transmittance of information therebetween. For example, and without limitation, this connection may be wired or wireless, direct, or indirect, and between two or more components, circuits, devices, systems, and the like, which allows for reception and/or transmittance of data and/or signal(s) therebetween. Data and/or signals therebetween may include, without limitation, electrical, electromagnetic, magnetic, video, audio, radio, and microwave data and/or signals, combinations thereof, and the like, among others. A communicative connection may be achieved, for example and without limitation, through wired or wireless electronic, digital, or analog, communication, either directly or by way of one or more intervening devices or components. Further, communicative connection may include electrically coupling or connecting at least an output of one device, component, or circuit to at least an input of another device, component, or circuit. For example, and without limitation, via a bus or other facility for intercommunication between elements of a computing device. Communicative connecting may also include indirect connections via, for example and without limitation, wireless connection, radio communication, low power wide area network, optical communication, magnetic, capacitive, or optical coupling, and the like. In some instances, the terminology “communicatively coupled” may be used in place of communicatively connected in this disclosure. In some embodiments, the processor 710 may include any computing device as described in this disclosure, including without limitation a microcontroller, microprocessor, digital signal processor (DSP) and/or system on a chip (SoC) as described in this disclosure. The processor 710 may include, be included in, and/or communicate with a mobile device such as a mobile telephone or smartphone. The processor 710 may include a single computing device operating independently, or may include two or more computing device operating in concert, in parallel, sequentially or the like. Two or more computing devices may be included together in a single computing device or in two or more computing devices. The processor 710 may interface or communicate with one or more additional devices as described below in further detail via a network interface device. Network interface device may be utilized for connecting the processor 710 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. The processor 710 may include but is not limited to, for example, a computing device or cluster of computing devices in a first location and a second computing device or cluster of computing devices in a second location. The processor 710 may include one or more computing devices dedicated to data storage, security, distribution of traffic for load balancing, and the like. The processor 710 may distribute one or more computing tasks as described below across a plurality of computing devices of computing device, which may operate in parallel, in series, redundantly, or in any other manner used for distribution of tasks or memory between computing devices. The processor 710 may be implemented using a “shared nothing” architecture in which data is cached at the worker, in an embodiment, this may enable scalability of system 700 and/or processor 710.

With continued reference to FIG. 7, processor 710 and/or a computing device may be designed and/or configured by memory 720 to perform any method, method step, or sequence of method steps in any embodiment described in this disclosure, in any order and with any degree of repetition. For instance, the processor 710 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. The processor 710 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Each of the components 710, 720, 730, and 740 may be interconnected, for example, using a system bus 750. The processor 710 is capable of processing instructions for execution within the system 700. In some implementations, the processor 710 is a single-threaded processor. In some implementations, the processor 710 is a multi-threaded processor. In some implementations, the processor 710 is a programmable (or reprogrammable) general purpose microprocessor or microcontroller. The processor 710 is capable of processing instructions stored in the memory 720 or on the storage device 730.

The memory 720 stores information within the system 700. In some implementations, the memory 720 is a non-transitory computer-readable medium. In some implementations, the memory 720 is a volatile memory unit. In some implementations, the memory 720 is a non-volatile memory unit.

The storage device 730 is capable of providing mass storage for the system 700. In some implementations, the storage device 730 is a non-transitory computer-readable medium. In various different implementations, the storage device 730 may include, for example, a hard disk device, an optical disk device, a solid-date drive, a flash drive, or some other large capacity storage device. For example, the storage device may store long-term data (e.g., database data, file system data, etc.). The input/output device 740 provides input/output operations for the system 700. In some implementations, the input/output device 740 may include one or more network interface devices, e.g., an Ethernet card, a serial communication device, e.g., an RS-232 port, and/or a wireless interface device, e.g., an 802.11 card, a 3G wireless modem, or a 4G/5G wireless modem. In some implementations, the input/output device may include driver devices configured to receive input data and send output data to other input/output devices, e.g., keyboard, printer and display devices 760. In some examples, mobile computing devices, mobile communication devices, and other devices may be used.

While this specification contains many specific implementation details, these should not be construed as limitations on the scope of what may be claimed, but rather as descriptions of features that may be specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous. Other steps or stages may be provided, or steps or stages may be eliminated, from the described processes. Accordingly, other implementations are within the scope of the following claims.

The phraseology and terminology used herein is for the purpose of description and should not be regarded as limiting.

The term “approximately”, the phrase “approximately equal to”, and other similar phrases, as used in the specification and the claims (e.g., “X has a value of approximately Y” or “X is approximately equal to Y”), should be understood to mean that one value (X) is within a predetermined range of another value (Y). The predetermined range may be plus or minus 20%, 10%, 5%, 3%, 1%, 0.1%, or less than 0.1%, unless otherwise indicated.

The indefinite articles “a” and “an,” as used in the specification and in the claims, unless clearly indicated to the contrary, should be understood to mean “at least one.” The phrase “and/or,” as used in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of or “exactly one of,” or, when used in the claims, “consisting of,” will refer to the inclusion of exactly one element of a number or list of elements. In general, the term “or” as used shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The use of “including,” “comprising,” “having,” “containing,” “involving,” and variations thereof, is meant to encompass the items listed thereafter and additional items.

Use of ordinal terms such as “first,” “second,” “third,” etc., in the claims to modify a claim element does not by itself connote any priority, precedence, or order of one claim element over another or the temporal order in which acts of a method are performed. Ordinal terms are used merely as labels to distinguish one claim element having a certain name from another element having a same name (but for use of the ordinal term), to distinguish the claim elements.

Having thus described several aspects of at least one embodiment of this invention, it is to be appreciated that various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

What is claimed is:

1. A system for remote rehabilitation of a movement disorder of a patient, comprising:
a wearable device comprising:
a sensor configured to generate movement data of a patient, the movement data indicative of one or more movement disorder symptoms of the patient;
a processor in communication with the sensor;
a stimulator in communication with the processor, wherein the processor is configured to cause the stimulator to stimulate a body part of the patient based on the movement data;
a wireless communication unit in communication with the processor; and
a virtual platform running on a server in communication with the wearable device via the wireless communication unit, the virtual platform programmed to receive the movement data from the wearable device and host both:
a provider application operable to run on a provider device, wherein the provider application is programmed to generate a therapy regime based on the movement data; and
a patient application operable to run on a patient device, wherein the patient application is programmed to compare the movement data to the therapy regime and provide real-time feedback to the patient through a patient device.

2. The system of claim 1, wherein the provider application is further programmed to generate a visualization of the movement data and display the visualization on the provider device.

3. The system of claim 1, wherein provider application is further programmed to:
receive parameter selections via the provider device; and
communicate the parameter selections to the wearable device via the virtual platform, wherein the processor of the wearable device is configured to command the stimulator based on the received parameter selections.

4. The system of claim 1, wherein the patient application is further programmed to provide an indication of an effectiveness of the therapy regime based on the comparison.

5. The system of claim 1, wherein the patient application is further programmed to guide the patient through one or more exercises based on the therapy regime.

6. The system of claim 5, wherein the patient application is further programmed to generate a completion score of the one or more exercises based on the movement data generated by the wearable device.

7. The system of claim 5, wherein the patient application is further programmed to present the patient with a visualization of the one or more exercises via the patient device.

8. The system of claim 1, wherein the real-time feedback provided by the patient application includes visualized directional guidance of the body part of the patient provided through the patient device.

9. The system of claim 1, wherein the stimulator is configured to deliver one of electrical, vibratory, or ultrasonic stimulation to the patient's body part.

10. The system of claim 1, wherein the stimulator provides stimulation output to one or more of Aδ fibers, C fibers, Ia afferents, Ib afferents, Aβ fibers, Aγ fibers, or dorsal root ganglion (DRG) neurons.

11. A method of remote rehabilitation of a movement disorder of a patient, comprising:
generating movement data by a sensor of a wearable device placed on a patient, the movement data indicative of one or more movement disorder symptoms of the patient;
communicating the movement data from a wireless communication unit of the wearable device to a virtual platform running on a server,
generating, by a provider application hosted by the virtual platform, a therapy regime based on the movement data;
comparing, by a patient application hosted by the virtual platform, the movement data to the therapy regime; and
providing, by the patient application, real-time feedback to the patient through a patient device in communication with the virtual platform based on the comparison.

12. The method of claim 11, further comprising:
receiving parameter selections via a provider device in communication with the provider application hosted by the virtual platform;
communicating the parameter selections to the wearable device via the wireless communication unit; and
commanding, by a processor of the wearable device, a stimulator of the wearable device to stimulate a body part of the patient based on the received parameter selections.

13. The method of claim 11, further comprising visualizing the movement data and providing the visualization to a provider device via the provider application in communication with the virtual platform.

14. The method of claim 11, further comprising indicating, by the patient application, an effectiveness of the therapy regime.

15. The method of claim 11, further comprising guiding the patient through one or more exercises based on the therapy regime via the patient device.

16. The method of claim 15, further comprising generating a completion score of the one or more exercises based on the movement data via the patient application.

17. The method of claim 16, further comprising presenting the patient with a visualization of the one or more exercises at the patient device via the patient application.

18. The method of claim 11, wherein the real-time feedback provided by the patient application includes visualized directional guidance of the body part of the patient provided through the patient device.

19. The method of claim 11, further comprising providing one or more of electrical, vibratory, or ultrasonic stimulation to the patient's body part.

20. The method of claim 11, further comprising providing stimulation output via a stimulator of the wearable device to one or more of Aδ fibers, C fibers, Ia afferents, Ib afferents, Aβ fibers, Aγ fibers, or dorsal root ganglion (DRG) neurons.
